(19) United States
(12) Patent Application Publication
(10) Pub. No.: US 2007/0218504 A1
(43) Pub. Date: Sep. 20, 2007

(54) HUMAN LEPTIN-DERIVED POLYPEPTIDES AND USES THEREOF

(75) Inventor: Allan Z. ZHAO, Sewickley, PA (US)

Correspondence Address: FOLEY AND LARDNER LLP;SUITE 500, 3000 K STREET NW, WASHINGTON, DC, 20007, US

(73) Assignee: UNIVERSITY OF PITTSBURGH

(21) Appl. No.: 11/683,555

(22) Filed: Mar. 8, 2007

Related U.S. Application Data
(60) Provisional application No. 60/780,281, filed on Mar. 9, 2006.

(51) Int. Cl. G01N 33/53; A61K 38/17; A61K 38/10; A61K 38/08; C07K 7/08; C07K 14/47

(52) U.S. Cl. 435/7.1; 514/12; 514/13; 514/14; 514/15; 514/16; 530/324; 530/325; 530/326; 530/327; 530/328; 530/329

(57) ABSTRACT

A polypeptide that contains an amino acid sequence present in human leptin blocks the inhibitory effect of human C-Reactive Protein on human leptin. Accordingly, such a polypeptide is implicated in an approach to treating or preventing conditions associated with the impact of CRP on leptin.

### STATEMENT REGARDING FEDERALLY SPONSORED RESEARCH OR DEVELOPMENT

Work related to the present invention had U.S. government support under Grant No. RO1DK064383-01, awarded by the National Institutes of Health. The government has certain rights in this invention.

### BACKGROUND OF THE INVENTION

The present invention relates to the prevention and treatment of conditions associated with C-Reactive Protein (CRP). More specifically, the invention relates to the utilization of human leptin-derived polypeptides for blocking the leptin-inhibitory effect of human CRP.

Documents cited in this description are denoted numerically, in parenthetical, by reference to a bibliography below.

Molecular and physiological evidence accumulated in the past decade has firmly established that leptin is a critical adipocyte hormone involved in regulation of energy intake and expenditure (1, 2). It is known that leptin acts primarily in specific regions of the brain, particularly the hypothalamus (50, 51). Leptin functions by signaling transduction through the central nervous system (CNS) to efficiently maintain a stable body weight, by its binding to the trans-membrane leptin receptor and subsequent stimulating proteins in the signaling pathways, such as signal transducers and activators of transcription (STAT), Janus kinases (JAK), and phosphatidylinositol-3 kinase (PI3-kinase) (12, 13).

The ratio of cerebrospinal leptin concentration to blood leptin concentration is lower in obese people than that in lean subjects (54, 55). Also, a null mutation in leptin or a leptin receptor gene can cause hyperphagia, severe obesity, and hyperglcemia (2-4). On the other hand, leptin replacement in leptin-deficient animals and humans can have profound normalizing effects on food intake and body weight.

Paradoxically, the majority of obese individuals have elevated rather than depressed levels of leptin (5). Therapeutic trials with exogenously administered leptin, which further raise leptin levels, have failed to induce meaningful weight loss (6). These observations present a conundrum, over the apparent ineffectiveness of leptin in preventing obesity, and have spawned the concept of “leptin resistance”(7, 8).

Recent studies have begun to elucidate potential molecular mechanisms. For example, elevation of suppressor of cytokine signaling-3 (SOCS-3), which is induced by leptin, might diminish leptin actions in the CNS and in pancreatic β-cells (8). Nevertheless, it remains unclear whether this mechanism can fully account for all leptin resistance.

C-reactive protein (CRP) is produced by the liver, in response to stress conditions, such as infection, trauma, and advanced cancer. Serum CRP level is generally elevated in obesity and is a marker of the low-grade inflammatory state associated with obesity and increased cardiovascular risk of obesity (9-11). Human CRP has been detected in the cerebral spinal fluid (CSF), evidencing that CRP can cross the blood-brain barrier (52). Furthermore, prior literature confirms that CRP can be produced in neurons as well (53). Elevation of CRP and its relationship to obesity and other disorders have yet to be adequately explained, however.

### SUMMARY OF THE INVENTION

It is an object of the present invention to provide a new approach to treating and preventing conditions associated with CRP, such as obesity, insulin resistance, diabetes, inflammation, metabolic syndromes, atherosclerosis, coronary artery disease, and infertility.

Accordingly, the present invention provides, in one aspect, a method for blocking the inhibitory effect of CRP on leptin to normalize the level of free leptin, by employing an agent to disrupt formation of CRP/leptin complexes. In one embodiment, the agent is a polypeptide comprised of a segment of 8-40 contiguous amino acids, which segment is present in full-length human leptin (SEQ ID NO: 1), exclusive of the core region and a segment of amino acids from human leptin residue 57 to residue 74 (SEQ ID NO: 2). In another embodiment, the group of polypeptides further excludes polypeptide E (SEQ ID NO: 5), discussed below. Subject to these qualifications, any polypeptide comprised of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acid residues may be used in this context. Preferably, the polypeptide is between 15 to 25 amino acid residues in length.

In accordance with another aspect of the present invention, polypeptides are provided that exhibit CRP/leptin complex-disruption activity. In one embodiment, a polypeptide of this category comprises the amino acid sequence of polypeptide B (SEQ ID NO: 3), or a sequence at least 60% identical to that of polypeptide B (SEQ ID NO: 3) with at least one modification. Other embodiments relate to a polypeptide comprising (i) the amino acid sequence of polypeptide C1 (SEQ ID NO: 4) or (ii) the amino acid sequence of polypeptide E (SEQ ID NO: 5), or a sequence at least 60% identical to these, respectively. The modifications contemplated in this regard are illustrated by but not limited to a conservative amino acid substitution, insertion or deletion, a C-terminal truncation, and a N-terminal truncation.

In accordance with a further aspect of the present invention, a method is provided for detecting CRP/leptin complex-disruption activity in an agent, comprising: (A) contacting the agent with CRP and leptin, in any order; and then (B) measuring an indicator selected from the group of CRP/leptin complex formation, amount of leptin, and amount of CRP, in the absence and presence of the agent, respectively, such that the indicator evidences the CRP/leptin complex disruption activity. In one embodiment, the amount of the CRP/leptin complex or the amount of CRP is measured by means of a leptin affinity column. In another embodiment, the amount of leptin is measured by assessing leptin activity, for example, by monitoring any component of leptin-stimulated signaling pathways, such as JAK2, STAT3, STAT5, STAT6, SH2 domain-containing protein tyrosine phosphatase (SHP-2), growth factor receptor binding-2 (Grb-2), extracellular-signal-regulated kinase (ERK), mitogen-activated protein kinase (MAPK) and PI-3 kinase.

In a related aspect, the invention provides a pharmaceutical composition comprising, with a pharmaceutically acceptable carrier, either a polypeptide with CRP/leptin complex-disruption activity or a nucleic acid encoding such a polypeptide. A pharmaceutical composition of the invention may be used to ameliorate a condition associated with CRP, including but not limited to obesity, inflammation, coronary heart disease, infertility, and diabetes. A composition of the invention also may be used to suppress food intake, to reduce body weight and adiposity, and to alleviate insulin resistance, respectively.

### BRIEF DESCRIPTION OF THE DRAWINGS

FIG. 1. Complete sequence of human leptin protein (SEQ ID NO: 1). Underlined sections represent sequences of polypeptide B (SEQ ID NO: 3), polypeptide Cl (SEQ ID NO: 4) and polypeptide E (SEQ ID NO: 5), from residues 26 to 50, 51 to 75 and 111 to 137, respectively.

FIG. 2A. Linear diagram of human leptin structure. Highlighted sections represent alpha-helix A from residues 25 to 44, alpha-helix B from residues 72 to 88, alpha-helix C from residues 92 to 115, alpha-helix D from residues 141 to 164, and helix E from residues 127 to 136, respectively.

FIG. 2B. Amino acid sequence alignment of human and mouse leptin protein. The conserved amino acids are shown as “*” in the sequence of mouse leptin, and the variable residues are shown in bold in both human an mouse leptin sequence. The underlined sequences represent the core region comprised of a-helices B, C and D.

FIG. 2C. Three-dimensional view of human leptin structure. The core region, comprised of α-helices B, C and D, is outlined.

FIG. 3. Purification of serum leptin interacting proteins (SLIPs) and identification of CRP. FIG. 3A shows five major SLIPs eluted of leptin affinity columns from human serum. From top to bottom on the silver-stained SDS gel, the bands correspond to SLIP-5, SLIP-4, SLIP-3, SLIP-2 and SLIP-1, with apparent molecular weight of 85, 70, 65, 42 and 30 Kd, respectively. Serum leptin is co-eluted with SLIPs, as pointed to by the dashed arrows. FIG. 3B confirms that SLIP-1 is human CRP with specific anti-CRP antibody in Western blot assay. Left lane represents the column fraction of SLIP-1, and right lane represents the human CRP standard.

FIG. 4A. Interference effect of human CRP on human leptin binding to its receptor. HEK293 cells stably transfected with the long-form human leptin receptor, OB-Rb (14), are used to examine whether human CRP interferes with the ability of human leptin to bind to its receptor. It is found that the Kd for human leptin and human leptin receptor on this cell line is 1.0×10⁻⁹ M. Pre-incubation of various amount of human CRP with ¹²⁵I-labeled human leptin reduces leptins' binding to its receptor in a dose-dependent manner. The IC₅₀ value is approximately 80 ng/ml of human CRP in the presence of about 2 ng/ml human leptin, which yields a molecular ratio of 5.8:1 (CRP:leptin, based on the pentameric structure of CRP). The dashed line at the bottom right of the graph indicates ¹²⁵I-leptin bound when two thousand fold excess of non-labeled leptin is added.

FIG. 4B. Effects of human CRP on mouse leptin. FIG. 4B shows that 2 ng/ml ¹²⁵I-labeled mouse leptin is pre-incubated with various amount of human CRP, before added to the HEK293 cells stably transfected with human OB-Rb. The IC₅₀ value is greater than 64 μg/ml of CRP in the presence of 2 ng/ml mouse leptin, which indicates that human CRP has much lower affinity toward mouse leptin than that toward human leptin. The dashed line at the bottom right of the graph indicates ¹²⁵I-leptin bound when two thousand fold excess of non-labeled mouse leptin is added.

FIG. 5. Determination of the effects of purified human or rat CRP on leptin signaling. FIGS. 5A and 5B illustrate the attenuating effects of human CRP (5A) and rat CRP (5B) on tyrosine phosphorylation of STAT3 induced by human- or murine-leptin, respectively, in a HEK293 cell line stably transfected with OB-Rb. FIG. 5C illustrates that human CRP does not have attenuation effect on tyrosin-phosphorylation of STAT3 induced by murine-leptin. FIGS. 5D and 5E illustrates the quantitative evaluation of the leptin-stimulated STAT3 activation in the presence of human CRP (5D) or rat CRP (5E) based on analysis of the digitally scanned images (such as those shown in 5A and 5B) with NIH-IMAGE 6.0 software. The data (relative fold of activation) represents the average of three different sets of experiments for human CRP, and two different sets for rat CRP, respectively. P<0.05 when compared to the leptin-only treated group.

FIG. 6. The effects of subcutaneous administration of human CRP on leptin-induced satiety and weight-reducing effects in ob/ob mice. Various amount of human CRP is either infused alone or together with human leptin at 0.3 mg/kg/day into 8-week old male ob/ob mice with osmotic pumps. The arrow indicates the day of surgery. Food intake (6A and 6C) and body weight (6B and 6D) are monitored daily throughout the infusion period of 6 days. Human CRP is infused at low dosage of 10 μg/day (6A & 6B) and high dosage (6C & 6D), respectively. Due to the variations in the body weight of ob/ob mice, body weight is expressed as a percentage of the pre-surgery body weight. In 6A and 6C, n=4 for each group except for saline and leptin group. In 6B and 6D, n=5 for the saline group, n=6 for the CRP- and leptin-infused, and n=7 for the group infused with leptin plus CRP. The symbos “*” and “#” indicate P<0.01 and P<0.05, respectively, in a tow-tailed Student's t-test. In both 6A and 6B, P<0.01 for leptin vs. saline and CRP from day 2; P<0.05 for leptin plus CRP vs. saline, vs. CRP, and vs. leptin group from day 3. In both 6C and 6D, P<0.01 or P<0.05 for leptin vs. saline, vs. CRP, and Vs. leptin plus CRP group form day 3. In addition, P<0.01 for leptin plus CRP vs. saline and vs. CRP in 6C on day 2.

FIG. 7. The effect of human leptin on CRP expression. Human hepatocytes are treated with physiological concentrations of human leptin for 24 hours before the medium is collected for Western assay of secreted CRP, and the results shown are quantified following the digital scanning of images of two different experiments.

FIG. 8. Western blot analysis of STAT3 activation as reflected by its tyrosine phosphorylation. Human CRP suppresses activation of STAT3 by leptin in the absence of polypeptide B. However, pre-incubation of human CRP with polypeptide B is able to block the inhibitory effect of human CRP on leptin signaling. The control indicates that polypeptide B itself does not have any effect on STAT phosphorylation.

FIG. 9. Western blot analysis of STAT3 activation as reflected by its tyrosine phosphorylation. Human CRP suppresses activation of STAT3 by leptin in the absence of polypeptide C1. However, pre-incubation of human CRP with polypeptide C1 is able to block the inhibitory effect of human CRP on leptin signaling. The control indicates that polypeptide C1 itself does not have any effect on STAT phosphorylation. As shown in FIG. 9A, the maximal effect of polypeptide C1 can be reached at a concentration as low as 50 nM, while in FIG. 9B, higher concentration of polypeptide C1 does not further improve its effect.

FIG. 10. Western blot analysis of STAT3 activation as reflected by its tyrosine phosphorylation. Human CRP suppresses activation of STAT3 by leptin in the absence of polypeptide E. However, pre-incubation of human CRP with polypeptide E is able to block the inhibitory effect of human CRP on leptin signaling. The control indicates that polypeptide E itself does not have any effect on STAT phosphorylation.

FIG. 11. Co-fusion of Peptide-E with human CRP and leptin through mini-osmotic pumps into the ob/ob mice restored the physiological effects of human leptin. Infusion dosages for human CRP and leptin were 48 μg/day/mouse and 12 μg/day/mouse, respectively. The infusion rate for Pep-E (P) was 24 μg/day/mouse (medium dosage, “m”) and 48 μg/day/mouse (high dosage, “h”). Food-intake (A and C) and body weight (B and D) were monitored daily throughout the infusion. The arrows in A-D indicate the day of surgery. At the end of infusion, blood samples were collected for the measurement of blood-glucose, human CRP, and human leptin. The serum concentrations of human CRP and human leptin were 0.85±0.1 μg/ml and 18 ng±2 ng/ml, respectively. FIG. 11E shows the blood glucose concentrations from the group of mice infused the high dosage of Pep-E (h). Also shown is glucose concentration from mice infused with human CRP and leptin but not with Pep-E.

FIG. 12. Western blot analysis of STAT3 activation measured by the degree of its tyrosine phosphorylation. As we have shown previously, co-incubation of human CRP with human leptin caused the reduction of leptin-stimulated STAT3 activation. However, the “hex-peptides” (L-form, NH₂—S—C—H-L-P—W—COOH; D-form, NH₂—W—P—L—H—C—S—COOH), when pre-incubated with human CRP and human leptin, failed to restore leptin-stimulated STAT3 activation (A). In the same set of experiment, co-incubation of Peptide-E (100, 200, 400, and 800 nM) with human CRP blocked the inhibitory effect of human CRP on leptin signaling (B). Peptide-E by itself did not induce any changes in STAT phosphorylation.

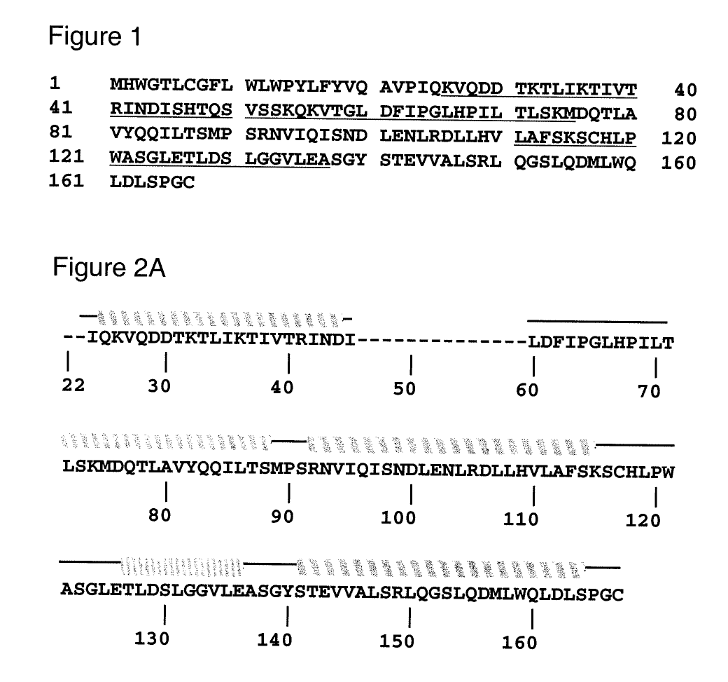

### DETAILED DESCRIPTION OF PREFERRED EMBODIMENTS

One aspect of the present invention relates to the inventor's discovery that leptin interacts with proteins in mammalian serum, so-called “Serum Leptin Interaction Proteins,” enumerated 1 through 5 (SLIPs 1-5). The inventor further determined that SLIP-1 is C-reactive protein (CRP).

The identification of CRP as a SLIP is remarkable in several respects. First, CRP is an inflammatory marker and generally increases in obesity (9, 15). An increase of CRP in association with obesity is consistent with the fact that inflammation is a metabolic complication of obesity, closely linked to central patterns of fat deposition and hepatic steatosis. Furthermore, elevated CRP in obesity and in insulin resistance is predictive of high risk for cardiovascular disease (11, 16).

The inventor has found that the physiological roles of CRP in energy balance are dependent on the presence of leptin. CRP alone does not influence food intake and body weight in the ob/ob mice, for instance.

Conversely, the inventor has discovered that CRP acts to abrogate leptin actions by directly binding to leptin. The formation of CRP/leptin complex impedes binding of leptin to its receptor and thereby interferes with leptin-mediated signal transduction. Consequently, the formation in vivo of CRP/leptin complex contributes to attenuation of the physiological effects of leptin, as manifested particularly in leptin resistance. The complex formation also underscores a mechanism by which CRP is understood to contribute directly to the pathogenesis of obesity and to its metabolic and cardiovascular complications.

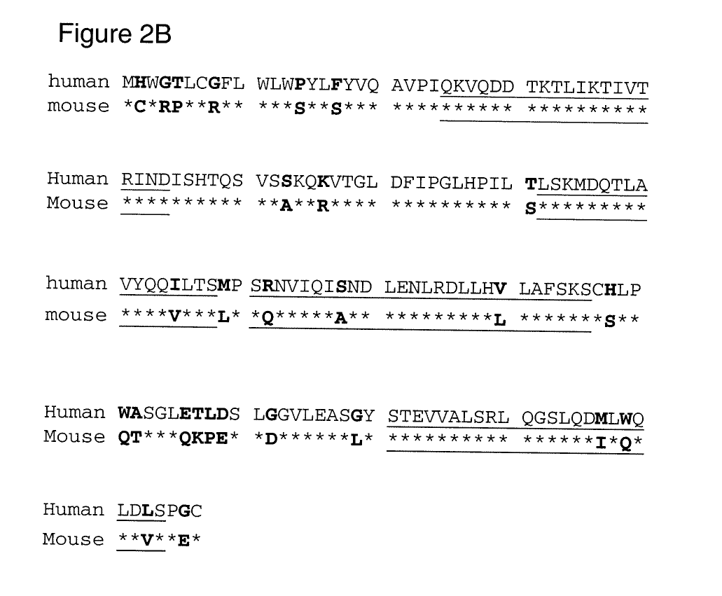

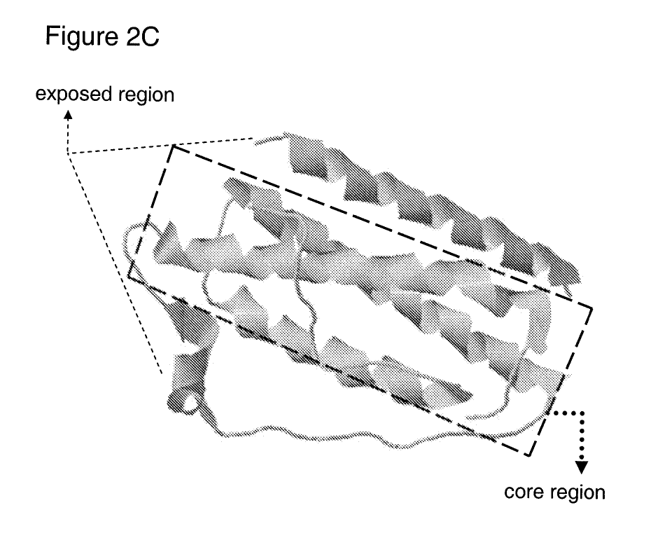

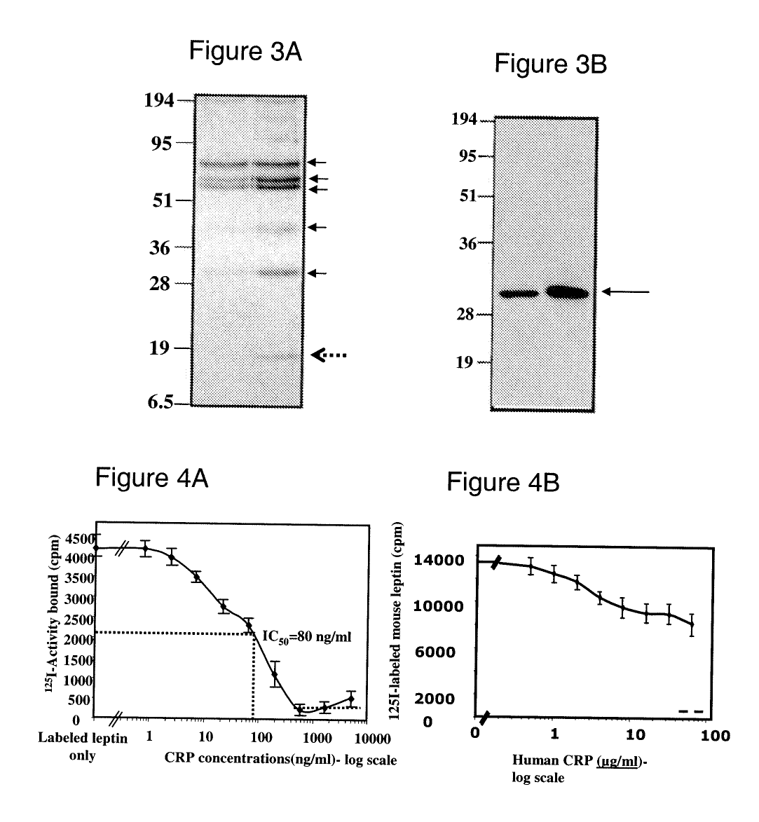

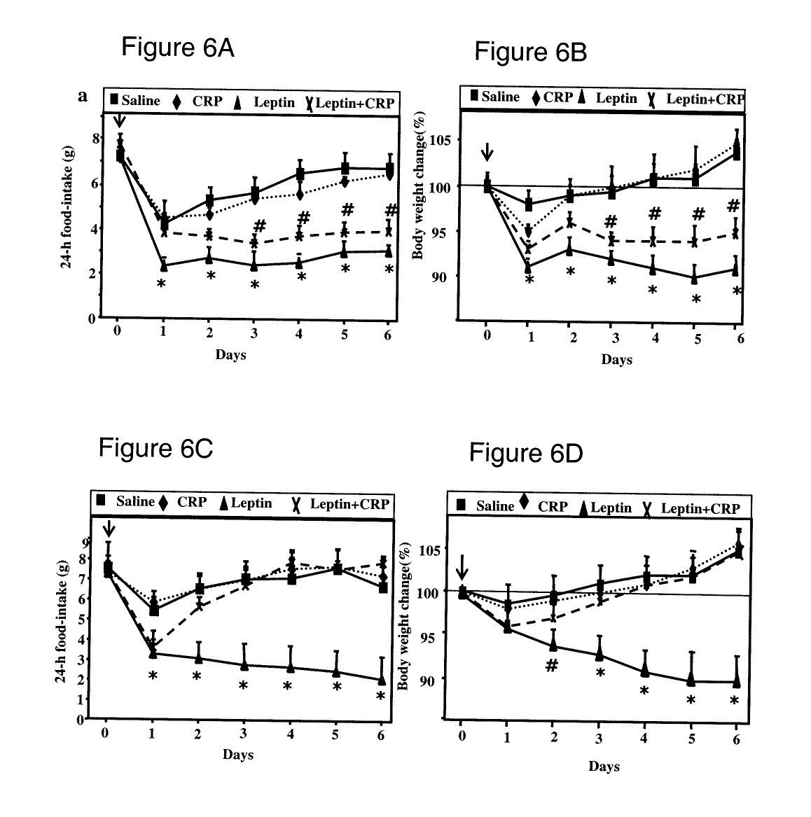

It also has been found that the role of CRP in leptin physiology is species-dependent. For example, FIG. 6 shows not only that human leptin binds to human CRP much more efficiently than to mouse leptin but also that human CRP has a more pronounced inhibitory effect on the signaling capacity of human leptin than on mouse leptin. A sequence alignment of human and mouse leptins (FIG. 2B) establishes that the flexible domains of leptins, which domains are free of the helix structures, are less conserved between the species.

These findings prompted the inventor's insight that a correlation exists between the leptin structure and its binding to CRP. Thus, a key aspect of the present invention relates to a correlation between the structure of human leptin and its formation of an in vivo complex with CRP.

More specifically, the inventor has shown that CRP interacts with critical domains of leptin that are required for binding to its receptor. From this perspective, leptin is seen to have (i) a core region of α-helices B, C, D, which is relatively inaccessible of the surface of the leptin molecule and (ii) α-helix A and other flexible domains that are exposed on the surface of the molecule (see FIG. 2C). Based on the understanding that the exposed region (ii) of leptin plays an important role in CRP binding, the present invention provides polypeptides of variable length (a) that have an amino acid sequence that corresponds to or is comprised of a sequence found in the exposed region of leptin and (b) that exhibit CRP/leptin complex-disruption activity, by competitively binding to the CRP.

Thus, the primary structure of a polypeptide of the present invention has a counterpart in those portions of the leptin molecule that do not include its core region (see FIG. 2C) and a N-terminal, 21-residue leader segment that is absent from the mature protein. As noted previously, one embodiment of the invention excludes from this genus of inventive polypeptides a polypeptide with an amino acid sequence consisting of those present in a segment from human leptin residue 57 to residue 74 (SEQ ID NO: 2). Another embodiment further excludes peptide E (SEQ ID NO: 5) from the genus.

Notwithstanding the above-stated qualifications, a polypeptide of the invention may embody, in its amino acid sequence, some overlap with the core region or the leader segment, so long as the presence of such portion of the polypeptide as corresponds to the overlap, for example, of 5 to 10 residues in either C- or N-terminal direction, does not substantially abrogate the CRP/leptin complex-disrupting activity of the polypeptide.

Illustrative of these polypeptides of the invention are three, designated B, C1, and E, the amino acid sequences of which appear below.

(SEQ ID NO: 3)

Polypeptide B: KVQDDTKTLIKTIVTRINDISHTQS

(SEQ ID NO: 4)

Polypeptide C1: VSSKQKVTGLDFIPGLHPILTLSKM

(SEQ ID NO: 5)

Polypeptide E: LAFSKSCHLPWASGLETLDSLGGVLEA.

Like other polypeptides of the invention, these are readily synthesized chemically and, when tested, display CRP/leptin complex-disruption activity.

This activity recommends the inventive polypeptides not only as drug candidates themselves but also as objects for conventional optimization and rational drug design, thereby to obtain drugs that block the inhibitory effect of CRP on leptin in vivo. Variants on these polypeptides should be effective to this end, for example.

The “variants” category in this context includes, inter alia, any polypeptide that displays both CRP/leptin complex-disruption activity and substantial homology to a polypeptide that corresponds to or comprises the sequence of a portion of the exposed region of leptin, as described above. In this regard, “substantial” denotes a homology at the amino acid level of at least about 60%, preferably about 70%, more preferably about 80%, and especially in the range between about 90% and 95%, as determined through a sequence-alignment comparison.

For such comparisons, various techniques are available for aligning sequences. Optimal alignment of sequences for comparison may be conducted by the local homology algorithm of Smith and Waterman (33), by the homology alignment algorithm of Needleman and Wunsch (34), and by the search for similarity method of Pearson and Lipman (35), for instance. Computerized implementations of these algorithms include but not limited to: CLUSTAL in the PC/Gene program by Intelligenetics, Mountain View, Calif.; GAP, BESTFIT, BLAST, FASTA, and TFASTA in the Wisc. Genetics Software Package, Genetics Computer Group (Madison, Wisc., USA), and the CLUSTAL program (36-40). See also 41-43.

A polypeptide that displays such substantial homology may by characterized by at least one conservative amino acid modification. A “conservative modification” to an amino acid sequence is a substitution, insertion, or deletion that does not eliminate the CRP/leptin complex-disruption activity that emblematic of a compound within the present invention.

It is known that conservative amino acid substitution may be made with a residue in the same class. In this regard, naturally occurring residues may be divided into classes, based on common side chain properties: (1) hydrophobic—Met, Ala, Val, Leu, Ile; (2) neutral hydrophilic—Cys, Ser, Thr; (3) acidic—Asp, Glu; (4) basic—Asn, Gln, His, Lys, Arg; (5) residues that influence chain orientation—Gly, Pro; and (6) aromatic—Trp, Tyr, Phe. Accordingly, one kind of conservative substitution replaces an amino acid at a given position with another within the same class.

In making conservative modifications, the hydropathic index of amino acids may be considered as well. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known that certain amino acids may be substituted for other amino acids that have a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is likewise understood that the substitution of like amino acids can be made effectively on the basis of hydrophilicity. The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (-0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, substitution is preferred of amino acids that have hydrophilicity values that are within ±2; those that are within ±1 are particularly preferred, and those within ±0.5 are especially preferred.

The following table enumerates exemplary conservative amino acid substitutions, in accordance with the principles discussed above.

TABLE I
Amino Acid Substitutions

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Heu, Val, Met, Ala, Phe, Norleucine | Leu |
| Leu | Norleucine, Ile, Val, Met, Ala, Phe | Ile |
| Lys | Arg, 1,4 Diaminobutyric acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tye |
| Tyr | Trp, Phe, Thr, Ser | Phe |
| Val | Ile, Met, Leu, Phe, Ala, Norleucine | Phe |

Thus, a conservative modification could entail the substitution of a native amino acid residue with a normative residue, such that there is little or no effect on the polarity or charge of the amino acid residue at that position. Alternatively, the involved substitution could introduce one or more non-naturally occurring amino acid residues, e.g., by solid-phase peptide synthesis or by means of genetic programming of a translational system (30).

By these and other approaches, which are known or which will be developed in the future, one can obtain variants that are peptidomimetic oligomers (31), which may display improved stability and/or bioavailability and, hence, represent better candidates for pharmaceutical purposes. In addition to those mentioned already, some modifications for this purpose (32) are: formation of cyclic variants, as by adding a C-terminal and an N-terminal cysteine with disulfide bond formation; amide nitrogen alkylation, e.g., for generation of N-methylated variants where one or more of the amide linkages in the peptide have a structure of C—N(CH₃)—CO—; side-chain modifications, e.g., methylation or halogenation of selected phenylalanine residues; substitution of methylalanine for alanine at selected sites; chirality modification, e.g., replacement of selected L-amino acids with D-amino acids; forming retro-inverse peptides, i.e., peptides with inverted sequence and opposite chirality (D instead of L); forming peptides with amide bond surrogates, e.g., selective reduction of —CO—NH— linkages to CH₂—NH— linkages; and replacing one or more alpha-carbons in the chain with nitrogen atoms, providing an azapeptide analogues.

Like other polypeptides within the invention, any given variant can be screened for CRP/leptin complex-disruption activity, as described below. Information gleaned in this fashion can be used in the design of other suitable variants. For example, if one discovered that a change to a particular amino acid residue resulted in destroyed, undesirably reduced, or unsuitable activity, variants with such a change would be avoided. Based on information gathered from such routine experiments and based on the information provided, in other words, one can readily determine the amino acids where further substitutions should be avoided, either singly or in combination with other changes.

Inventive polypeptides and variants can be “PEGylated,” a term that connotes derivatization by covalently binding of one or more molecules of polyethylene glycol (PEG). In various media, particularly aqueous media, the long, chain-like PEG molecule is hydrated and in rapid motion. The motion of the PEG substituent sweeps out a large volume and prevents the approach and interference of other molecules. The PEG polymer chain can protect the molecule from immune responses and other clearance mechanisms, thereby prolonging and preserving bioavailability of the molecule. By the same token, biological absorption, distribution, and clearance can be modified by altering the size, weight, shape, and linkage used to connect the PEG strand to the molecule in question (29).

In accordance with this invention, disruption of the formation of CRP-leptin complex is evaluated as a therapeutic tool for treating or preventing conditions associated with a dysfunction in a leptin-affected pathway. Pharmacological intervention influencing the association between human CRP and leptin, as described above, could be used to block the inhibitory effects of human CRP on leptin, and could lead to suppression of food intake and reduction of body weight and adiposity, as well as alleviation of obesity, metabolic syndromes, inflammation, atherosclerosis, infertility, insulin resistance, and type II diabetes. In addition, one could alter the interaction between human CRP and leptin, pursuant to the invention, to increase transport of leptin into the central nervous system (CNS), thus enhancing leptin-induced effects in the CNS.

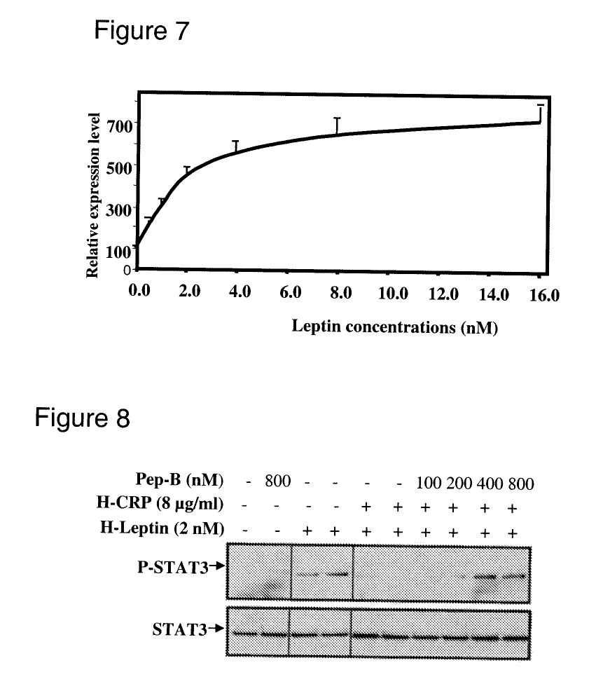

Recent studies have indicated a positive role of circulating leptin in regulating CRP production (15, 26-28), and CRP in turn can bind leptin and dampen the action of leptin in the CNS and periphery. The inventor has discovered that, although human leptin, within the physiological range, produces a dose-dependent positive effect on CRP secretion, sustained increase of leptin does not result in higher production of CRP (see FIG. 7). Thus, polypeptides of the present invention may be used as a therapeutic agent to liberate leptin from CRP/leptin complex formation, consequently restoring the physiological functions of leptin.

Accordingly, this invention also relates to a pharmaceutical composition comprised of the polypeptide with CRP/leptin complex-disruption activity or the nucleic acid encoding the polypeptide, plus a pharmaceutically acceptable carrier therefor, as well as to administering the composition to a subject in an amount effective to counter the CRP-mediated diminution of leptin that is available to induce its normal signaling pathways (“free leptin”).

In an embodiment that involves the use of a polypeptide-encoding nucleic acid, considerations that pertain to the field of gene therapy become relevant. For example, these considerations would inform (i) constructing a mammalian expression vector, encoding a polypeptide as described here, and (ii) introducing the vector in the context of a therapeutic protocol, according to the invention. Vector transfer technologies for use in this regard are illustrated by: (a) direct DNA microinjection into cells, in vivo or ex vivo; (b) ballistic gold particle delivery; (c) liposome-mediated transfection; (d) receptor-mediated gene transfer; and (e) the use of DNA transposons (44-46). In addition, the present invention comprehends the use of an adenovirus-based gene therapy system for delivery of a polypeptide-encoding nucleotide, as mentioned. The construction and packaging of adenoviral vectors are well known techniques, and potentially useful adenoviral vectors are described, for example, in U.S. Pat. No. 5,707,618 and in cited publications (47-49).

As mentioned above, a pharmaceutical composition of the invention could be used to treat a subject with any condition associated with the impact of CRP on leptin. Consequently, the modes of administration for a pharmaceutical composition of the invention are those suitable for administering a therapeutic polypeptide or protein.

Thus, a pharmaceutical composition of the invention could be formulated for oral, ocular, nasal, nasolacrimal, topical (e.g., pulmonary, sublingual, huccal), or intrathecal (cerebrospinal fluid) delivery, in accordance with accepted medical practice. By the same token, formulation of a composition of the invention would accommodate the mode of administration, among other considerations, to include in the pharmaceutically acceptable carrier, as appropriate: excipients, such as starch, lactose, crystalline cellulose, calcium lactate, magnesium aluminometasilicate, and anhydrous silicate); disintegrators, e.g., carboxymethylcellulose and calcium carboxymethylcellulose; lubricants, illustrated by magnesium stearate and talc; coating agents such as, hydroxyethylcellulose; and flavoring agents of the sort used for oral and mucosal formulations. For external agents would be used, in conventional manner, solubilizers and auxiliary solubilizers capable of forming aqueous injections (e.g., distilled water for injection, physiological saline, and propylene glycol), suspending agents (e.g., surfactant such as polysorbate 80), pH regulators (e.g., organic acid and metal salt thereof) and stabilizers are used for injections; and aqueous or oily solubilizers and auxiliary solubilizers (e.g., alcohols and fatty acid esters), tackifiers (e.g., carboxy vinyl polymer and polysaccharides) and emulsifiers.

Another aspect of the invention concerns detecting CRP/leptin complex-disruption activity in an agent, such as a polypeptide or variant as described above, a mimetic of such a polypeptide or some other small molecule. In a preferred embodiment, the inventive methodology comprises (A) contacting the agent with CRP and leptin, in any order; and then (B) measuring an indicator selected from the group of CRP/leptin complex formation, amount of leptin, and amount of CRP, in the absence and presence of the agent, respectively, such that the indicator evidences the CRP/leptin complex disruption activity.

Agents can be screened by various means. The screening can be performed by incubating leptin, concurrently or sequentially, with CRP and a putative agent, to be tested for complex-disruption activity. This may be effected by loading a leptin affinity column with a fixed amount of CRP, in the presence or absence of an agent with potential complex-disruption activity, and then identifying those that interact with CRP in such a way that they preclude or diminish CRP/eptin complex formation. An agent with complex-disruption activity should increase the amount of CRP washed through the column, relative to an equivalent run in the absence of the agent.

Alternatively, a potential active agent could be identified based on interaction with CRP to inhibit or preclude CRP/leptin complex formation. This might involve first contacting CRP and the agent with potential complex-disruption activity to form CRP-agent complex, removing unbound agent, contacting the resulting CRP with leptin, measuring CRP-leptin complex formation, for example, by immunoprecipitation and radio-labeling, and comparing that value with CRP leptin complex formation in the absence of the agent.

An agent with complex-disruption activity also could be identified by screening for an ability to inhibit the signaling capabilities of leptin in cultured cells. For example, a potential agent can be co-incubated with leptin and CRP before the mixture is incubated with the cultured cells containing leptin receptors. Subsequently, leptin-induced activity of STAT3 or PI3-kinase, etc. can be assayed. The results would be compared to those obtained in the absence of the agent. An active agent should revitalize the ability of leptin to signal in the cultured cells. In this context, the assay could involve monitoring any component of leptin-stimulated signaling pathways, such as JAK2, STAT3, STAT5, STAT6, SH2 domain-containing protein tyrosine phosphatase (SHP-2), growth factor receptor binding-2 (Grb-2), extracellular-signal-regulated kinase (ERK), mitogen-activated protein kinase (MAPK), and PI-3 kinase.

Pursuant to the invention, screening for an agent with complex-disruption activity could be effected on a high-throughput basis. To this end, for example, the BEK293 cells over-expressing OB-Rb may be grown in a 96-well plate. The HEK293 cells also could carry a leptin-responsive reporter gene, such as a leptin-inducible promoter operatively linked to a luciferase reporter. The responsiveness of the cells to leptin exposure can induce phosphorylation of STAT3, which is detectable by fluorescence-labeled antibody or by an increase in luciferase activity in a fluorescent plate reader described in previous publications. Pre-incubation of CRP with leptin would negate the effects of leptin, while a polypeptide with CRP/leptin complex-disruption activity would restore the effects of leptin.

In another embodiment, screening of potential agents that are disruptive to the interaction of CRP and leptin is achieved through co-administration of a putative agent with leptin, in the presence or absence of CRP, directly into the CNS, e.g., by intracerebralventricular injection (see Example SB, infra). Pursuant to this aspect of the invention, the effectiveness of the agent in restoring the satiety and weight-reduction actions of leptin can be evaluated, for instance, by measurement of food-intake and body weight. In this context, such measurement would be the indicator for evidencing CRP/leptin complex disruption activity.

The detailed description of the present invention continues by reference to the following examples, which are illustrative only and not limiting of the invention.

### EXAMPLE 1

### Purification and Identification of Serum Leptin-Interacting Proteins

A. Purification of SLIPs

Mouse or human recombinant leptin (from AF Parlow of NHPP, Torrance, CA) was covalently linked to Sepharose-beads with an Amino-Link kit (Pierce Biotechnology, Rockford, Ill.). Rat or human serum (1.5 ml) was loaded onto the affinity column, allowed to pass through the resin, and the column was then washed with 15-volumes of PBS-0.5% Tween-20 (for rat samples), or a Ca²⁺-containing buffer (0.1M Tris-Cl, 0.1 M NaCl, 2 mM CaCl₂) (for human serum samples). Retained material was eluted with an acidic glycine solution, and the eluate was immediately neutralized in a Tris-buffer (50 mM, pH=9.5).

Five major SLIPs were identified from the elute of human leptin-affinity columns on a silver-stained SDS gel with apparent molecular weight of 30-, 42-, 65-, 70-, and 85-Kd, correspondingly designated as the human SLIP-1, 2, 3, 4, and 5. Serum leptin could also be co-eluted with SLIPs. All five human SLIPs have rat counterparts since passage of rat serum through the mouse leptin-affinity column yielded proteins of very similar molecular weights.

B. Identification of SLIP-1

Rat SLIP-1 was excised from an SDS-PAGE gel and subjected to MALDI-TOF assay following tryptic digestion. This analysis identified rat SLIP-1 as rat C-reactive protein (CRP). Similarly, human SLIP-1 was also identified as human CRP in a mass-spec analysis (Nano-LC-MS/MS in-gel protein identification). The mass-spec assay was performed on Thermo Finnigan LTQ mass spectrometer by CTO-BIO Services (Rockville, Md.). Data analysis was carried out with the Turbo-Sequest software. A filtered score of >2.33 for a doubly charged peptide (“z”) indicates a significant match with human CRP. SLIP-1 is further confirmed as CRP with polyclonal antibodies that specifically recognize human or rat CRP in Western blot assays.

### EXAMPLE 2

### Demonstration of Direct Binding of CRP and Leptin by Immuno-Precipitation Assay

To explore a physical interaction between CRP and leptin, the direct binding of these proteins was determined by an immuno-precipitation assay. Rat CRP was purified from fresh rat serum to >95% purity employing a previously established affinity-purification protocol (20). This degree of purity was comparable to that of a human CRP preparation from a commercial source The purity was further confirmed by mass spectrometry. The purified human- and rat-CRP proteins were pre-mixed with recombinant human and mouse leptin, respectively, before addition of antibodies specific for human- and mouse-leptin. The concentrations of CRP and leptin in the precipitation reaction were all within the physiological ranges that have been observed in humans or rats (11 & 15). In parallel experiments, recombinant leptin also was pre-mixed with human- or rat-serum to allow for direct interaction prior to immuno-precipitation. The protein precipitates obtained were subjected to Western blot assays using specific anti-CRP antibodies. Immuno-precipitation using anti- human leptin could pull down human CRP from both the leptin/CRP mixture and from human serum. Similarly, immuno-precipitation using anti mouse-leptin could bring down rat CRP from the leptin/CRP mixture as well as from rat serum. Direct interaction of CRP and leptin is further confirmed, where immuno-precipitation was performed using anti human-CRP and rat CRP-antibodies and was found to pull down leptin proteins.

### EXAMPLE 3

### Demonstration of CRP Interference on Leptin Binding to its Receptor

To examine if CRP binding interferes with the stability of human leptin to bind to its receptors, a HEK293 cell line stably transfected with the long-form human leptin receptor, OB—Rb, was used (17). Human leptin was iodinated with Na¹²⁵I using the lodogen method. Briefly, 15 μg of recombinant human leptin in 100 mM Phosphate buffer pH 7.5 were incubated with 1 mCi of carrier-free Na¹²⁵I (2200 Ci/mmol) in a glass tube containing 50 μg Iodogen. After 10-minute incubation at room temperature, the reaction was stopped with 100 μl 0.1% trifluoroacetic acid (TFA). The reaction mixture was immediately purified by reverse-phase HPLC. The separation employed a 5-minute isocratic step at 20% eluant B in A, followed by two consecutive 30 min linear gradients from 20 to 50%, then from 50 to 60% eluant B in A (where eluant A is water containing 0.1% TFA and eluant B is acetonitrile containing 0.1% TFA) at a flow rate of 1.5 ml/min. The ¹²⁵I-labeled leptin (˜2 ng/ml) was pre-incubated with various concentrations of human CRP in 1 ml of μ-MEM for 1 hour before added to the OBR-expressing HEK293 cells. The incubation would last for 3 hour at 4° C. before the media was aspirated and the cells were dissolved in 0.1N NaOH for scintillation counting.

It was found that the K_{d} for leptin and its receptors on this cell line was 1.0×10⁻⁹M. As shown in FIG. 4A, pre-incubation of human CRP with ¹²⁵I-labeled human leptin reduced leptin's binding to its receptors in a dose-dependent manner. The IC₅₀ value was approximately 80 ng/ml of CRP in the presence of about 2 ng/ml human leptin, which yielded a molecular ratio of 5.8:1 (CRP:leptin, based on the pentameric structure of CRP). On the other hand, human CRP displayed lower affinity towards mouse leptin (IC₅₀ greater than 64 μg/ml in the presence of about 2 ng/ml mouse leptin, as shown in FIG. 4B.

### EXAMPLE 4

### Signaling Studies

To determine if the interaction between CRP and leptin dampens the cellular actions of leptin, the ability of leptin to stimulate tyrosine phosphorylation of STAT3 and PI3-kinase activity was assessed in the presence of CRP in vitro. HEK293 cells over-expressing OB-Rb were serum starved for 2 hours before addition of leptin and CRP (CHEMICON or EMD Biosciences). Leptin, in the presence or absence of CRP, was pre-incubated in μ-MEM (with additional 1 mM Ca²⁺) for 30 minutes at 37° C. before being applied to the cells. After a 30-minute incubation with leptin, the cells were harvested in lysis buffer (18). The resultant protein extract was subjected to a Western blot analysis using an anti-phospho-STAT3 specific antibody (Cell Signaling, Beverly, Mass.). For studies involving primary hypothalamic neurons, the hypothalamus from rats was surgically isolated immediately after euthanasia, and placed in the DMEM for incubation with leptin in the presence or absence of CRP.

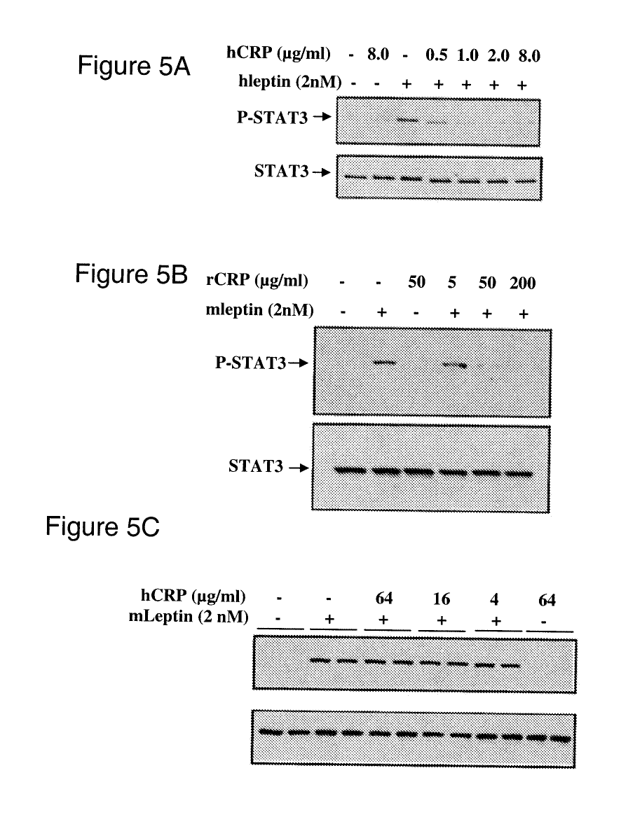

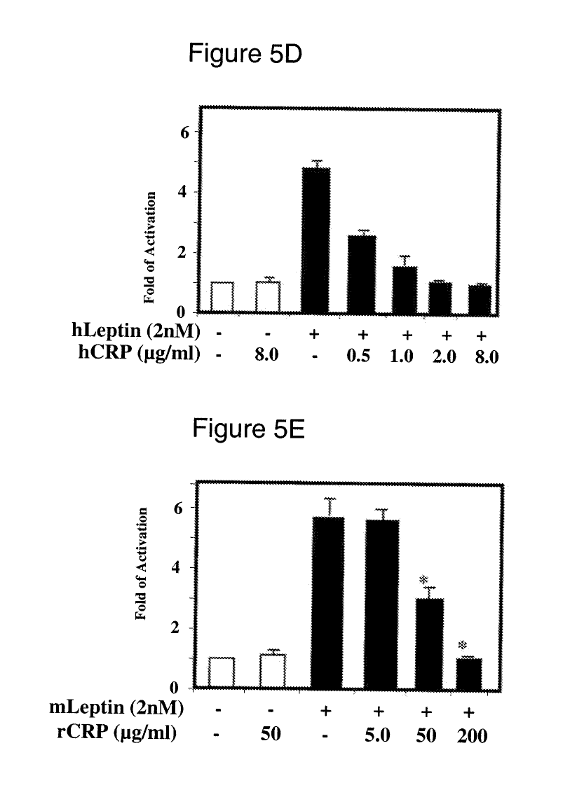

Both human CRP and rat CRP were found to attenuate the tyrosine-phosphorylation of STAT3 induced by human- or murine-leptin, respectively (FIGS. 5A & 5B). The concentrations of human CRP and rat CRP required to block leptin-induced STAT3 phosphorylation are within the ranges observed in the human- and rat-plasma (11, 21 & 22), although higher amounts of rat-versus human-CRP were required to achieve equivalent effects. Consistent with the observation of low affinity between human CRP and mouse leptin, high concentrations of human CRP were unable to block mouse leptin-induced STAT3 activation (FIG. 5C).

Further studies revealed that the attenuation of leptin signaling by human CRP requires the presence of calcium ions, since addition of excess EGTA to the medium blocked the inhibitory effects of human CRP on leptin signaling.

More phosphor-STAT3 and PI3-kinase assays have demonstrated that human CRP specifically inhibited leptin signaling in primary hypothalamic neurons, since parallel assays show that human CRP, at an even higher concentration, did not suppress IL-6 induced activation of STAT3 in human primary hepatocytes. Similarly, rat CRP did not block insulin-stimulated PI3-kinase activity in 3T3-L1 adipocytes.

Human hepatocytes were isolated from donor livers. Briefly, the livers were perfused in a three-step process. A calcium-free buffer supplemented with EGTA is used to break intracellular junctions, which is followed by the same perfusion buffer without EGTA. Finally, a calcium-containing buffer supplemented with digestive enzymes (collagenase and DNase) was used. Following digestion, parenchymal hepatocytes were isolated by three steps of low-speed centrifugation. Cells with a viability of at least 85% were plated and cultured in hepatocytes maintenance medium (HMM) (Cambrex BioScience, Walkersville, Md.) supplemented with 10% fetal bovine serum (FBS), 0.1 μM dexamethasone, 0.1 μM insulin, and 50 μg/ml gentamicin on collagen-coated tissue culture dishes for 2-4 hours. The cells were subsequently cultured in the above medium without FBS.

Approximately 24 hours after seeded, the hepatocytes were washed with serum-free HMM supplemented with 0.1 μM dexamethasone and 0.1 μM insulin, and then cultured in the same medium for another 48 hours. Subsequently, the cells were switched to serum-free HMM supplemented with 0.1 μM dexamethasone before stimulated with leptin (NHPP) or rhIL-6 (R&D Systems, Minneapolis, Minn.) for 24 hours or 48 hours. For experiments involving PI3-kinase inhibitors, hepatocytes were pretreated with LY294002 (EMD Biosciences, Calif.) for 1 hour before hormonal stimulation.

In a separate control, it has been found that human serum albumin, even at concentrations of several hundred-fold higher than human CRP, had no appreciable effect on leptin-induced STAT3 phosphorylation. Similarly, human serum amyloid P component (SAP), circulating at much higher concentrations than CRP, also did not influence leptin signaling when co-incubated in cultured cells.

### EXAMPLE 5

### Assessment of CRP Functions and Monitoring Physiological Indicators

The functional effects of human CRP were evaluated to see whether elevated circulating CRP via infusion would be able to negate or attenuate the suppression of food intake and loss of weight that are normally evoked by leptin. Ob/ob mouse, in lieu of rat model, was adopted in the experiments due to its minimum basal plasma level of CRP (23)

A. Attenuation of Human Leptin by Subcutaneously Administration of Human CRP

Micro-osmotic pumps (#1007D, DURECT, Cupertino, Calif.) were subcutaneously implanted into ob/ob or wild-type mice according to the instructions of the manufacturer. The osmotic pumps were pre-filled with saline, CRP, leptin or leptin plus CRP. Food intake (24-hour), body weight, and body temperature were monitored daily post-surgery. Blood glucose concentrations were measured using tail-vein blood samples using a Precision Plus® glucose meter, product of Medisense (Abbot Park, Ill.).

Human- and mouse-leptin concentrations were measured using ELISA kits from CHEMICON (Temecular, Calif.) and R&D Systems (Minneapolis, Minn.), respectively, accordingly to the instructions of the manufacturers. Serum mouse insulin and human CRP were determined with ELISA kits from LINCO Research (St. Charles, Mo.) and HELICA (Fullerton, Calif.), respectively. Serum- and tissue-triglycerides were determined with the protocols described previously (19).

Various amounts of human CRP, either alone or with human leptin, were administered via micro-osmotic pumps implanted s.c. into 8-week old ob/ob mice. With an ELISA assay that can detect both free and bound forms of leptin, the serum leptin concentrations were found to be almost identical in the leptin- and leptin/CRP-infused animals. During the 6-day continuous infusion period, human leptin produced the expected reduction in food-intake and body weight in the ob/ob mice, as shown in FIGS. 6A-6D. Although co-infusion of human CRP at a low infusion dosage at 10 μg/day only partially attenuated these effects of human leptin, at a higher concentration of 40 μg/day, human CRP was able to completely block the actions of leptin to restrain appetite and induce weight loss.

Consistent with these observations, there was also attenuation of leptin-induced energy expenditure as gauged by recordings of body temperature. Human CRP, at the high infusion dosage of 40 μg/day, completely blocked the increase of rectal temperature induced by leptin.

Importantly, the serum concentrations of human CRP attained in these mice are similar to the physiological range observed in human plasma from ostensibly healthy donors.

Although leptin administration to ob/ob mice alleviated diabetes, including the lowering of blood glucose, plasma insulin, and serum- and hepatic triglyceride, co-infusion of human CRP has reverted these effects. As a further confirmation of the negative effects of CRP on leptin actions, activation of STAT3 in the hypothalamic tissues of ob/ob mice was also inhibited by co-infusion of CRP with leptin. By contrast, administration of human CRP alone did not affect food intake or body weight. Thus, the impact of CRP is dependent upon the presence of leptin.

The above in vivo data were harvested from mice experiencing infusions of a mixture of CRP and leptin stored in a single mini-osmotic pump. Human CRP and leptin were also infused in separate osmotic pumps. The total human leptin concentrations in the sera of all groups infused with leptin (leptin only or leptin plus CRP) were very comparable to each other (15-20 ng/ml). For reasons not yet clear, with the separate pump approach, higher infusion dosages of human CRP were required to achieve serum concentrations of human CRP in ob/ob mice comparable to those realized with the single pump approach. Also compared to infusion from a single pump, detectable effects of human CRP on leptin were realized later, likely due to the time required to achieve diffusion and binding equilibrium. Nevertheless, at serum concentrations that matched those achieved with a single osmotic pump, human CRP was again found to attenuate the physiological actions of human leptin, including reductions of food intake and body weight as well as correction of blood glucose and serum insulin.

To complement the in vivo studies further, the satiety and weight-reducing functions of human leptin were evaluated in mice producing transgenically expressed human CRP (24). The average baseline serum concentration of human CRP in these mice was approximately 15 μg/ml. Despite such high basal levels, human CRP alone did not meaningfully affect the food intake and body weight of the transgenic mice, consistent with the low affinity of human CRP towards mouse leptin. To evaluate the effects of human leptin in the transgenic mice, a dose of 0.6 mg/kg/day, which has been known to maximally but only temporarily affected energy balance in wild-type mice (25), was infused. Infusion of human leptin into wild-type littermates of CRP transgenics produced the expected reductions in food intake, body weight, and epidydimal fat-pad weight. Nevertheless, these physiological effects of human leptin were completely blunted in the CRP-transgenic mice. At the end of infusion, the total human leptin concentration in the sera of transgenic mice averaged at 7.6 ng/ml, which was higher than that in the wild-type littermates at 3.2 ng/ml. These results indicate a negative effect of human CRP on the physiological actions of leptin.

B. Attenuation of Human Leptin by Direct Intracerebralventricular (i.c.v.) Injection of Human CRP

The results from subcutaneous administration of human CRP indicate that CRP is not only a marker for obesity-related co-morbidities but also is itself involved in the regulation of adiposity, through direct interaction with leptin and subsequent induction of leptin resistance. Intracerebralventricular injection of human CRP into the CNS of ob/ob mice further demonstrates that CRP can attenuate the actions of leptin within the CNS.

Male C57BL/6J ob/ob mice used in this study were obtained from the Jackson Laboratory, Bar Harbor. Mice (7-8 weeks of age) were fed standard chow and water ad libitum, housed in individual cages under controlled temperature at 22° C. and a 12-hour light-dark cycle with light from 0700 to 1900 h. Food intake and body weight were measured daily throughout the study. All experimental procedures were performed according to protocols approved by the Institutional Animal Care and Use Committee at the University of Pittsburgh.

Cannulas were surgically implanted into the lateral ventricles of male ob/ob mice. Specifically, the mice were intraperitoneally anesthetized with 250 mg/Kg Avertin (Sigma-Aldrich). A plastic cannula was inserted into lateral cerebral ventricle: −0.7 mm relative to bregma, 1.3 mm lateral of midline, and 2 mm down to the calvarium. The exteriorized cannula was secured on the skull with original Super Glue. To prevent the cannula from becoming blocked by blood clots, a 28-gauge stainless syringe pinhead was inserted into each cannula. The animals were allowed to recover for 1 week after surgery until the food-intake reaches at least 90% of the pre-operative level.

All agents were injected slowly with a volume of 1 μl using a 5 μl syringe (Hamilton, Reno, Nev.). For instance, human CRP was injected at a lower concentration of 1.2 μg per animal and a higher concentration of 2.3 μg per animal into the lateral ventricles. The i.c.v injection of human CRP was followed 24 hours later by injection of human leptin at a sub-maximal dose of 100 ng/animal. In a parallel experiment, human leptin was also injected via i.c.v. following a pre-injection of PBS. In each injection scheme, food was returned to the animals 1 hour after the injection, and the amount of food-intake and body weight were measured 24 hours later.

The hypothalamic tissues were surgically dissected right after sacrificing the mice. A portion of each tissue was extracted for protein in a protein lysis buffer. The activation of STAT3 was evaluated using Western blot assays with an antibody against phospho-tyrosine-STAT3 as described above (Cell Signaling, Beverly, Mass.). The membrane was striped and applied with an antibody against STAT3 to evaluate the overall level of STAT3 (Cell Signaling, Beverly, Mass.).

All paired comparisons were subject to a two-tailed Student t-test with P<0.05 considered statistically significant.

The results obtained by i.c.v. injection are confirmatory of those obtained by subcutaneous administration. Thus, the animals injected with CRP alone consumed essentially the same amount of food when compared to those injected with PBS alone, again indicating that CRP alone was not able to influence the energy balance in the ob/ob mice. Injection of human leptin led to a significant decrease in food-intake by 40.2% (P<0.001), and reduction of body weight by 2.17% (P<0.001). In contrast, pre-injection of human CRP with the dosage of 1.2 μg partially blunted the satiety effect and weight reducing effects of human leptin; while at a higher concentration of 2.3 μg/μl, human CRP completely blocked these actions of leptin.

Consistent with the results in Example 5A, these data indicate that human CRP attenuates the effects of human leptin in a dosage dependent manner. Moreover, i.c.v. injection of human CRP did not influence the satiety and weight-reducing effects induced by mouse leptin. Mouse leptin was injected via i.c.v following a pre-injection of PBS or human CRP. The experiments demonstrated a 32.9% vs 32.4% in decrease of food intake (P<0.05) , as well as a 1.80% vs 1.76% in reduction of body weight (P<0.05) for ob/ob mice pre-injected with PBS and human CRP, respectively.

Additionally, human leptin was injected via i.c.v. for an extended period of two days subsequent to a pre-injection of human CRP with a dosage of 2.3 μg/injection. In a parallel experimental scheme, daily injection of human leptin was performed for two consecutive days subsequent to a pre-injection of PBS. Following the PBS treatment, daily injection of human leptin for two days led to gradual decreases in food intake and body weight. In contrast, pre-injection of human CRP completely blunted the satiety and weight-reducing actions of the first injection of human leptin, and at least partially blocked the effects of the second injection of human leptin.

To determine whether the interaction between human CRP and leptin dampens the cellular actions of leptin, an assessment was made of the ability of leptin to stimulate tyrosine phosphorylation of STAT3 in the hypothalamic tissues. As described above, human CRP at 2.3 μg/injection or PBS was pre-injected via i.c.v., followed by an injection of human leptin or mouse leptin, each at 100 ng/injection. Subsequently, the hypothalamic tissues were dissected and immediately placed into liquid nitrogen for a rapid freezing. A portion of the frozen tissue was homogenized in a protein lysis buffer for the extraction of proteins. The protein extract was then subjected to Western blot assay as described above.

Again, injection of human leptin, following the PBS injection, induced a sharp increase in the level of phosph-STAT3, while pre-injection of human CRP drastically diminished the activation of STAT3 induced by human leptin. Consistent with the physiological data presented above, pre-injection of human CRP did not block mouse leptin-induced STAT3 activation.

### EXAMPLE 6

### Assessment of Leptin's Effect on CRP Production

In order to test if leptin itself might stimulate the expression of CRP in hepatocytes, CRP secretion and expression were analyzed.

Hepatocytes were isolated and treated as described in Example 4. Culture medium was centrifuged to remove detached cells and then heated at 95° C. for 5 minutes in SDS-sample buffer (Boston Bioproducts, Mass.). After separation on 10% SDS polyacrylamide gels, proteins were electro-transferred overnight to nitrocellulose membranes. Pre-blocked using 5% non-fat milk in PBS with 0.5% Tween 20, the membranes were incubated with appropriate primary antibody for 2 hours at room temperature, washed and incubated for 1 hour with a corresponding peroxidase-conjugated anti-CRP antibody at room temperature. To detect proteins, the membranes were incubated with ECL reagent and the blots were exposed to X-Omat film (Kodak). Two kinds of CRP antibodies were used: anti-human CRP (EMD Biosciences, Calif.) and anti-rat CRP (Alpha Diagnostic Intl., San Antonio, Tex.).

CRP mRNA expression was measured by quantitative real-time RT-PCR. In brief, total hepatocyte RNA was isolated with TRIzol reagent (Invitrogen, Carlsbad, Calif.), treated with DNase I (Ambion, Austin, Tex.), and reverse-transcribed using a Suprescript kit (Invitrogen, Carlsbad, Calif.). The real-time PCR reaction was performed with a Taqman machine (ABI7700) as follows: 50° C., 2 minutes, 1 cycle; 95° C., 10 minutes, 1 cycle; 95° C., 15 seconds, 60° C., 1 minute, 40 cycles. The sequences of probes and primes are as follows:

Human CRP probe:

(SEQ ID NO: 6)

5′-/6 FAM/TGCAAGGCGAAGTGTTCACCAAACC/BHQ_A/-3′;
Human CRP 5′ primer:

(SEQ ID NO: 7)

5′-GGCGGGCACTGAAGTATGAA-3′;
Human CRP 3′ primer:

(SEQ ID NO: 8)

5′-GCCTCAGGGCCACAGCT-3′;
Human 18s rRNA probe:

(SEQ ID NO: 9)

5′-/6-FAM/CGCGCAAATTACCCACTCCCGA/BHQ_A/-3′;
Human 18s rRNA 5′ primer:

(SEQ ID NO: 10)

5′-ACATCCAAGGAAGGCAGCAG-3′;
Human 18s rRNA 3′ primer:

(SEQ ID NO: 11)

5′-TCGTCACTACCTCCCCGG-3′.
Rat 18s rRNA probe:

(SEQ ID NO: 12)

5′-/6-FAM/CGCGCAAATTACCCACTCCCGA/BHQ_A/3′;
Rat 18s rRNA 5′ primer:

(SEQ ID NO: 13)

5′-GCACGAGGCGAGAAAGGA-3′;
Rat 18s rRNA 3′ primer:

(SEQ ID NO: 14)

5′-TTCGTCACTACCTCCCCGG-3′;
Rat CRP probe:

(SEQ ID NO: 15)

5′-/6-FAM/CCTTCTTGGGACTGATGCTGGTGACA/BHQ_A/-3′;
Rat CRP 5′ primer:

(SEQ ID NO: 16)

5′-TGTGCCACCTGGGAGTCTG-3′;
Rat CRP 3′ primer:

(SEQ ID NO: 17)

5′-TTCCGCACCCTGGGTTT-3′.

Although a short treatment of 6-8 hours did not influence CRP gene expression, incubation of human hepatocytes with human leptin at physiological concentrations for 24 hours produced a dose-dependent positive effect on secreted CRP in the culture medium (FIG. 7). A parallel examination of CRP gene expression, using real-time PCR assay, also showed a dosage-dependent effect on human CRP mRNA expression. Pre-incubation of human primary hepatocytes with a specific PI3-kinase inhibitor, LY294002, completely blocked the effect of leptin on CRP expression, suggesting that leptin-induced hepatic production of CRP is a PI3-kinase dependent process.

### EXAMPLE 7

### Assessment of Polypeptides

To determine the domains that are relevant to the interaction between human leptin and human C-reactive protein, a series of polypeptides (average length of 25 amino acids) were chemically synthesized. Polypeptide B (SEQ ID NO: 3), polypeptide C1 (SEQ ID NO: 4) and polypeptide E (SEQ ID NO: 5) are representatives of such peptides.

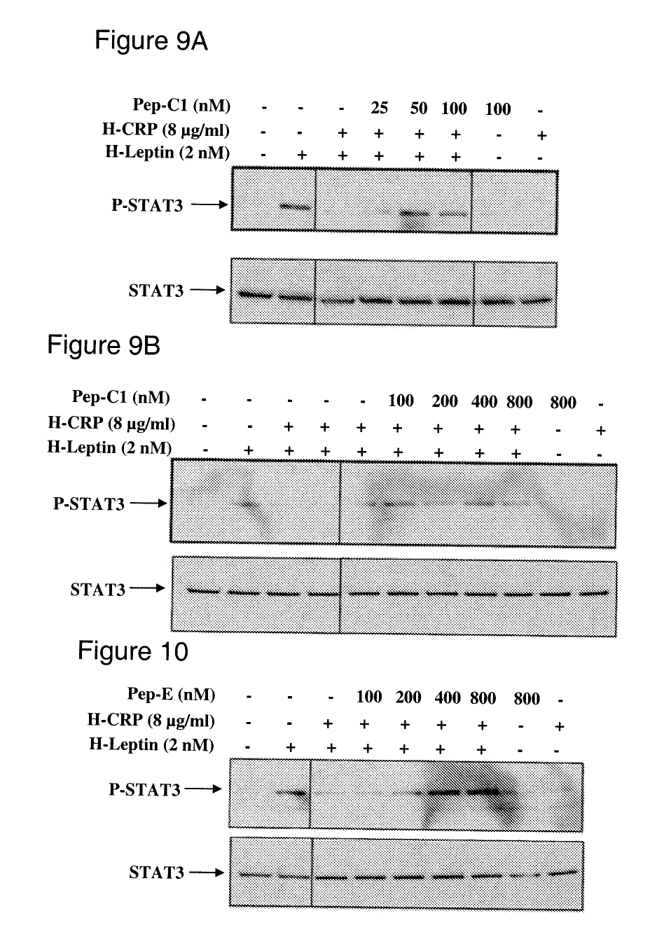

In the in vitro experiment, each of these polypeptide was pre-incubated with human CRP for 30 minutes before being mixed with human leptin for an additional 45 minutes. The resultant mixture was then added to the HEK-293 cells over-exprssing the long form leptin receptors, OB-Rb. After a 30-minute incubation, the cells were harvested in a protein extraction buffer (18). The signaling of leptin in the cells, measured by its ability to stimulate tyrosine phosphorylation on STAT3 in a western blot assay, was evaluated (see FIGS. 8, 9, 10 for polypeptides B, C1 and E, respectively). Human CRP suppressed the activation of STAT3 by leptin, but pre-incubation of each of the polypeptide with human CRP was able to block the inhibitory effect of human CRP and to restore leptin signaling. Each of the polypeptide by itself did not induce any changes in STAT phosphorylation. It was also observed that polypeptide C1 is more effective at a lower concentration in blocking the CRP activity on leptin.

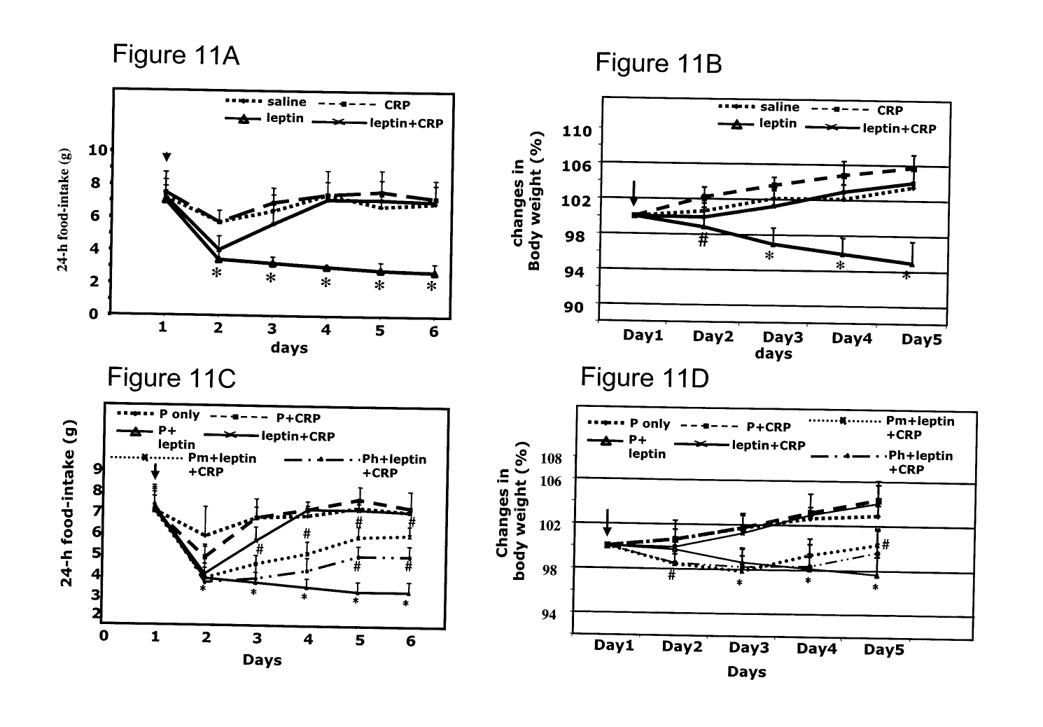

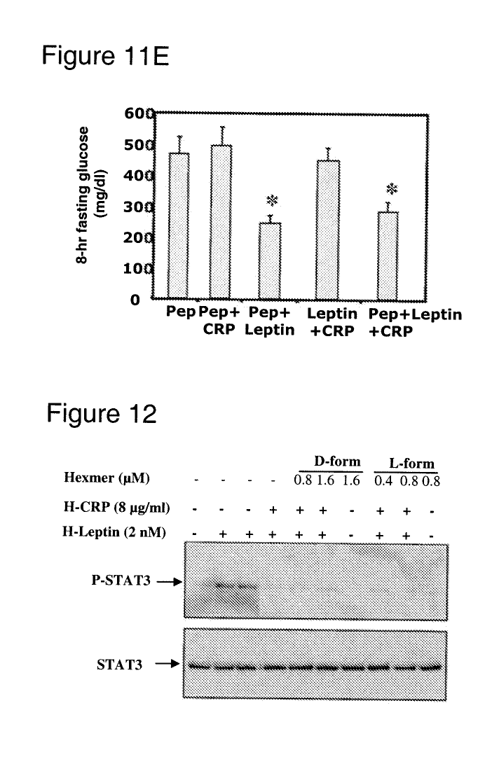

In the in vivo experiment, polypeptide E was co-infused through mini-osmotic pumps with human CRP and leptin into the ob/ob mice. Food intake in the period of 24 hours and body weight were followed during the infusion period (FIGS. 11A-11D). Although in the absence of the polypeptide, human CRP was able to block the satiety and weight-reducing effects of human leptin, co-fusion of polypeptide E with human CRP and leptin was able to restore both of these functions of human leptin. At the end of infusion, it was found that polypeptide E was able to restore the beneficial effect of human leptin on blood glucose (FIG. 11E).

### EXAMPLE 8

### Comparison of Polypeptides with the Hexamer of U.S. Pat. No. 6,777,388

Pursuant to an in vitro protocol similar to that described in Example 6, a hexamer disclosed in U.S. Pat. No. 6,777,388, NH₂—S—C—H-L-P—W—COOH (SEQ ID NO:18), was pre-incubated with human CRP (8 μg/ml) and human leptin (2 nM) for 1 hour. The resultant mixture then was added to HEK-293 cells that over-expressed the long form leptin receptor, OB—Rb. After a 30-minute incubation, the cells were harvested in a protein extraction buffer. To evaluate signaling of human leptin in the cells, tyrosine phosphorylation on STAT3 was measured in a western blot assay.

The results (FIG. 12) show that the hexamer of the '388 patent, at concentrations as high as 0.8 μM (or 800 nM), was unable to block the negative effect of human CRP on the signaling capability of human leptin. In the same set of experiments, polypeptide E was able to restore the signaling capability of human leptin.

### Cited Publications

Each of the following publications and each publication cited above is incorporated herein, in its entirety, by reference.
1. Friedman, J. M. & Halaas, J. L. Leptin and the regulation of body weight in mammals, Nature 395, 763-770 (1998).
2. Ahima, R. S. & Flier, J. S. Leptin, Annu. Rev. Physiol. 62, 413-437 (2000).
3. Zhang, Y. et al. Positional cloning of the mouse obese gene and its human homologue, Nature 372, 425-432 (1994).
4. Lee, G. H. et al. Abnormal splicing of the leptin receptor in diabetic mice, Nature 379, 632-635 (1996).
5. Schwartz, M. W. et al. Evidence that plasma leptin and insulin levels are associated with body adiposity via different mechanisms, Diabetes Care 20, 1476-1481 (1997).
6. Heymsfield, S. B. et al. Recombinant leptin for weight loss in obese and lean adults: a randomized, controlled, dose-escalation trial, J.A.M.A. 282, 1568-1575 (1999).
7. Gura, T. Obesity research, Leptin not impressive in clinical trial, Science 286, 881-882 (1999).
8. Bjorbaek, C., El-Haschimi, K., Frantz, J. D. & Flier, J. S. The role of SOCS-3 in leptin signaling and leptin resistance, J. Biol. Chem. 274, 30059-30065 (1999).
9. Maachi, M. et al. Systemic low-grade inflammation is related to both circulating and adipose tissue TNFα, leptin and IL-6 leels in obese women, Int. J. Obes. Relat. Metab. Disord. 28, 993-997 (2004).
10. Willerson, J. T. & Ridker, P. M. Inflammation as a cardiovascular risk factor, Circulation 109 (21 Suppl 1), II2-10 (2004).
11. Schulze M. B. et al. C-reactive protein and incident cardiovascular events among men with diabetes, Diabetes Care 27, 889-894 (2004).
12. Considine R. V. Human leptin: an adipocyte hormone with weight-regulatory and endocrine functions, Semin. Vasc. Med. 5, 15-24 (2005).
13. Frühbeck G. Intracellular signaling pathways activated by leptin, Biochem. J. 393, 7-20 (2006).
14. Vaisse, C. et al. Leptin activation of STAT3 in the hypothalamus of wild-type and ob/ob mice but not db/db mice, Nat. Genet. 14, 95-97 (1996).
15. Kazumi, T., Kawaguchi, A., Hirano, T. & Yoshino, G. C-reactive protein in young, apparently healthy men: associations with serum leptin, QTc interval, and high-density lipoprotein-cholesterol. Metabolism 52, 1113-1116 (2003).
16. Ridker, P. M. High-sensitivity C-reactive protein, inflammation, and cardiovascular risk: from concept to clinical practice to clinical benefit. American Heart Journal 148, S19-26 (2004).
17. Friedman-Einat, M. et al. Serum leptin activity in obese and lean pateients. Regul Pept. 111, 77-82 (2003).
18. Zhao, A. Z., Zhao, H., Teague, J., Fujimoto, W. & Beavo, J. A. Attenuation of insulin secretion by insulin-like growth factor 1 is mediated through activation of phosphodiesterase 3B, Proc. Natl. Acad. Sci. USA 94, 3223-3228 (1997).
19. Huan, J. N. et al. Adipocyte-selective reduction of the leptin receptors induced by antisense RNA leads to increased adiposity, dyslipidemia, and insulin resistance. J. Biol. Chem. 278, 45638-45650 (2003).
20. Tseng, J. & Mortensen, R. F. Binding of human C-reactive protein (CRP) to plasma fibronectin occurs via the phosphorylcholine-binding site, Mol. Immunol. 25, 679-686 (1988).
21. Aronson, D. et al. Obesity is the major determinant of elevated C-reactive protein in subjects with the metabolic syndrome, Int. J. Obes. Relat. Metab. Disord. 28, 674-679 (2004).
22. de Beer, F. C. et al. Isolation and characterization of C-reactive protein and serum amyloid P component in the rat, Immunology 45, 55-70 (1982).
23. Zahedi, K. et al. Major acute-phase reactant synthesis during chronic inflammation in amyloid-susceptible and -resistant mouse strains, Inflammation 15, 1-14 (1991).
24. Szalai, A. J. & McCrory, M. A. Varied biologic functions of C-reactive protein: lessons learned from transgenic mice, Immunol. Res. 26, 279-287 (2002).
25. Harris, R. B. et al. A leptin dose-response study in obese (ob/ob) and lean (+/?) mice, Endocrinology 139, 8-19 (1998).
26. Shamsuzzaman, A. S. et al. Independent association between plasma leptin and C-reactive protein in healthy humans. Circulation 109, 2181-2185 (2004).
27. Hukshorn, C. J. et al. Leptin and the proinflammatory state associated with human obesity, J. Clin. Endocrinol. Metab. 89, 1773-1778 (2004).
28. Mackintosh, R. M. & Hirsch, J. The effects of leptin administration in non-obese human subjects, Obes. Res. 9, 462-469 (2001).
29. Roberts, M. J. et al. Chemistry for peptide and protein PEGylation, Advanced Drug Delivery Reviews, 459-476 (2002).
30. Forster, A. C. et al. Programming peptidomimetic syntheses by translating genetic codes designed de novo, Proc. Nat'l. Acad. Sci. USA 100, 6353-6357 (2003).
31. Patch, J. A. & Barron, A. E. Mimicry of bioactive peptides via non-natural, sequence-specific peptidomimetic oligomers, Curr. Opn. Chem. Biol. 6, 872-877 (2002).
32. Adessi, C. & Soto, C. Converting a peptide into a drug: Strategies to improve stability and bioavailability, Curr. Medicinal Chem. 9, 963-978 (2002).
33. Smith & Waterman, Adv. Appl. Math. 2: 482 (1981).
34. Needleman & Wunsch, J. Mol. Biol. 48: 443 (1970).
35. Pearson & Lipman, Proc. Natl. Acad. Sci. USA 85: 2444 (1988).
36. Higgins & Sharp, Gene 73: 237-244 (1988).
37. Higgins & Sharp, CABIOS 5: 151-153 (1989).
38. Corpet et al., Nucleic Acids Research, 16: 10881-90 (1988).
39. Huang et al., Computer Applications in the Biosciences 8: 155-165 (1992).
40. Pearson et al., Methods in Molecular Biology 24: 307-331 (1994).
41. McGinnis S., & Madden, T. L. BLAST: at the core of a powerful and diverse set of sequence analysis tools, Nucleic Acids Res. 32:W20-W25 (2004).
42. Altschul, S. F. et al. Issues in searching molecular sequence databases, Nature Genet. 6:119-129 (1994)
43. Altschul, S. F. A protein alignment scoring system sensitive at all evolutionary distances, J. Mol. Evol. 36:290-300 (1993).
44. Morgan, R. A. & Anderson, W. F., Annu. Rev. Biochem. 62: 191-217 (1993).
45. Ivics, Z., Cell 91: 501-510 (1997).
46. Boulay, J -L. & Récipon, H., Curr. Opin. Biotechnol. 9: 445-450 (1998).
47. Jozkowicz, A. & Dulak, J. Helper-dependent adenoviral vectors in experimental gene therapy, Acta Biochim. Polonica 52: 589-599 (2005).
48. Antinozzi, P. A. et al., Ann. Rev. Nutr. 19: 511-544 (1999).
49. Verma, I. M. & Somia, N., Nature 389: 239-242 (1997).
50. Woods, S. C., Seeley, R. J., Porte, D. Jr. & Schwartz, M. W., Signals that regulate food intake and energy homeostasis, Science 280, 1378-1383 (1998).
51. Elmquist, J. K., Maratos-Flier, E., Saper, C. B. & Flier, J. S., Unraveling the central nervous system pathways underlying responses to leptin, Nat. Neurosci. 1, 445-450 (1998).
52. Rajs G., Finzi-Yeheskel Z., Rajs A. & mayer M., C-reactive protein concentrations in cerebral spinal fluid in gram-positive and gram-negative bacterial meningitis, Clin. Chem. 48, 591-592 (2002).
53. Yasomjima K., Schwab C., McGeer E. G. & McGreer P. L., Human neurons generate C-reactive protein and amyloid P: upregulation in Alzheimer's disease, Brain Res. 887, 80-89 (2000).
54. Schwartz M. W., Peskind E., Raskind M., Boyko E. J. & Porte D. Jr., Cerebrospinal fluid leptin levels: relationship to plasma levels and to adiposity in humans, Nat. Med. 2, 589-593 (1996).
55. Caro J. F. et al., Decreased cerebrospinal-fluid/serum leptin ration in obesity: a possible mechanism for leptin resistance, Lancet 348, 159-161(1996).

What is claimed is:

1. A polypeptide comprising a segment of 8-40 amino acid residues that are present, as a contiguous sequence, in the human leptin protein sequence (SEQ ID NO: 1) exclusive of a core consisting of α-helices B, C and D, wherein (i) said polypeptide disrupts formation of CRP/leptin complex and (ii) the amino acid sequence of said polypeptide is exclusive of SEQ ID NO: 2.

2. The polypeptide according to claim 1, wherein said segment is present in an exposed surface region of said human leptin protein.

3. The polypeptide according to claim 1, comprising a segment of 15-25 amino acid residues.

4. The polypeptide according to claim 2, comprising:
(A) the amino acid sequence of SEQ ID NO: 3; or
(B) the amino acid sequence of SEQ ID NO: 3, with at least one modification that is (i) a conservative amino acid substitution, insertion, or deletion, (ii) a C-terminal truncation, or (iii) a N-terminal truncation, wherein the polypeptide is at least 60% identical to the amino acid sequence of SEQ ID NO: 3.

5. The polypeptide according to claim 2, comprising:
(A) the amino acid sequence of SEQ ID NO: 4; or
(B) the amino acid sequence of SEQ ID NO: 4, with at least one modification that is (i) a conservative amino acid substitution, insertion, or deletion, (ii) a C-terminal truncation, or (iii) a N-terminal truncation, wherein the polypeptide is at least 60% identical to the amino acid sequence of SEQ ID NO: 4.

6. The polypeptide according to claim 2, comprising:
(A) the amino acid sequence of SEQ ID NO: 5; or
(B) the amino acid sequence of SEQ ID NO: 5, with at least one modification that is (i) a conservative amino acid substitution, insertion, or deletion, (ii) a C-terminal truncation, or (iii) a N-terminal truncation, wherein the polypeptide is at least 60% identical to the amino acid sequence of SEQ ID NO: 5.

7. A pharmaceutical composition comprising (i) the polypeptide of claim 1 or the nucleic acid encoding said polypeptide and (ii) a pharmaceutically acceptable carrier therefor.

8. A method for detecting CRP eptin complex disruption activity in an agent, comprising:
(A) contacting said agent with CRP and leptin, in any order; and then
(B) measuring an indicator selected from the group of CRP/leptin complex formation, amount of leptin, and amount of CRP, in the absence and presence of said agent, respectively,
wherein said indicator evidences said CRP/leptin complex disruption activity.

9. The method according to claim 8, wherein step (B) comprising loading a leptin affinity column with said CRP in the absence and presence of said agent, respectively, and then measuring the amount of CRP washed through.

10. The method according to claim 8, wherein step (B) comprising measuring leptin activity by STAT3 and/or PI3-kinase assay.

11. A method for normalizing the level of free leptin in a subject, comprising administering to said subject a pharmacologically effective amount of a pharmaceutical composition of claim 7.

12. A method according to claim 11, wherein said subject suffers from or is at risk of a condition selected from the group consisting of obesity and type II diabetes.

13. A method according to claim 11, wherein said subject suffers from or is at risk of a condition selected from the group consisting a metabolic syndrome, inflammation, atherosclerosis, and infertility.


SEQUENCE LISTING (20 sequences)

SEQ ID NO: 1 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 2 - protein, 18 aa, Homo sapiens
VTGLDFIPGLHPILTLSK

SEQ ID NO: 3 - protein, 25 aa, Homo sapiens
KVQDDTKTLIKTIVTRINDISHTQS

SEQ ID NO: 4 - protein, 25 aa, Homo sapiens
VSSKQKVTGLDFIPGLHPILTLSKM

SEQ ID NO: 5 - protein, 27 aa, Homo sapiens
LAFSKSCHLPWASGLETLDSLGGVLEA

SEQ ID NO: 6 - DNA, 25 bp, Artificial Sequence
  Description of Artificial Sequence Synthetic probe
tgcaaggcgaagtgttcaccaaacc

SEQ ID NO: 7 - DNA, 20 bp, Artificial Sequence
  Description of Artificial Sequence Synthetic primer
ggcgggcactgaagtatgaa

SEQ ID NO: 8 - DNA, 17 bp, Artificial Sequence
  Description of Artificial Sequence Synthetic primer
gcctcagggccacagct

SEQ ID NO: 9 - DNA, 22 bp, Artificial Sequence
  Description of Artificial Sequence Synthetic probe
cgcgcaaattacccactcccga

SEQ ID NO: 10 - DNA, 20 bp, Artificial Sequence
  Description of Artificial Sequence Synthetic primer
acatccaaggaaggcagcag

SEQ ID NO: 11 - DNA, 18 bp, Artificial Sequence
  Description of Artificial Sequence Synthetic primer
tcgtcactacctccccgg

SEQ ID NO: 12 - DNA, 22 bp, Artificial Sequence
  Description of Artificial Sequence Synthetic probe
cgcgcaaattacccactcccga

SEQ ID NO: 13 - DNA, 18 bp, Artificial Sequence
  Description of Artificial Sequence Synthetic primer
gcacgaggcgagaaagga

SEQ ID NO: 14 - DNA, 19 bp, Artificial Sequence
  Description of Artificial Sequence Synthetic primer
ttcgtcactacctccccgg

SEQ ID NO: 15 - DNA, 26 bp, Artificial Sequence
  Description of Artificial Sequence Synthetic probe
ccttcttgggactgatgctggtgaca

SEQ ID NO: 16 - DNA, 19 bp, Artificial Sequence
  Description of Artificial Sequence Synthetic primer
tgtgccacctgggagtctg

SEQ ID NO: 17 - DNA, 17 bp, Artificial Sequence
  Description of Artificial Sequence Synthetic primer
ttccgcaccctgggttt

SEQ ID NO: 18 - protein, 6 aa, Artificial Sequence
  Description of Artificial Sequence Synthetic peptide
SCHLPW

SEQ ID NO: 19 - protein, 6 aa, Artificial Sequence
  Description of Artificial Sequence Synthetic peptide
WPLHCS

SEQ ID NO: 20 - protein, 167 aa, Mus musculus
MCWRPLCRFLWLWSYLSYVQAVPIQKVQDDTKTLIKTIVTRINDISHTQSVSAKQRVTGLDFIPGLHPILSLSKMDQTLAVYQQVLTSLPSQNVIQIANDLENLRDLLHLLAFSKSCSLPQTSGLQKPESLDGVLEASLYSTEVVALSRLQGSLQDILQQLDVSPEC